(19) United States
(12) Patent Application Publication
(10) Pub. No.: US 2014/0099657 A1
(43) Pub. Date: Apr. 10, 2014

(54) Methods for bacillus spore preparation

(71) Applicant: Tony L. Buhr, King George, VA (US)

(72) Inventor: Tony L. Buhr, King George, VA (US)

(73) Assignee: United States Government, as represented by the Secretary of the Navy, Arlington, VA (US)

(21) Appl. No.: 13/136,900

(22) Filed: Aug. 5, 2011

(51) Int. Cl. C12N 1/20; C12Q 1/02

(52) U.S. Cl. 435/29; 435/252.5

(57) ABSTRACT

A preparation method for strains of Bacillus including Bacillus anthracis, Bacillus anthracis V770-NPI-R, and Bacillus thuringiensis using an aerated media of a protein-rich sporulation broth inoculated with a live Bacillus strain. The inoculated protein-based sporulation broth is incubated, centrifuged and washed to isolate high-purity spores.

### STATEMENT OF GOVERNMENT INTEREST

The invention described herein was made in the performance of official duties by one or more employees of the Department of the Navy, and the invention herein may be manufactured, practiced, used, and/or licensed by or for the Government of the United States of America without the payment of any royalties thereon or therefore.

### FIELD OF INVENTION

The present invention relates in general to the field of microbial culture preparations and in particular to the preparation of Bacillus spores in broth culture.

Terminology

As used herein, the term “agitation” means agitation or mixing of protein-based broth using a shaker or stirrer (e.g., stir-bar, mixer, or paddle). Agitation further requires that air must be exchanged through an air-exchange membrane (e.g., filtered cap). Exchange of oxygen or ambient air can be passive or oxygen and air can be forced into the protein-based broth under pressure.

As used herein, the terms “protein-based broth,” “protein-based sporulation broth” and “medium” mean a microbial growth medium consisting of greater than 0.1% amino acids, peptides or proteins, and having an amendment of less than 0.1% simple sugars (e.g., glucose). Protein-based broths may include, but are not limited to, soy broth; acid or enzyme digests of protein-rich substrates such as case in, beef extract, yeast extract; and peptones. Protein-based broths may include a manganese supplement (e.g., MnCl₂).

As used herein, the term “single inoculation” means one inoculation permitting sporulation so that there are a total of fifty or fewer cell divisions.

### BACKGROUND OF THE INVENTION

Bacillus spores from various species/strains are used as biological indicators (BI) to monitor sterilization efficacy after decontamination of medical equipment, autoclaving, buildings, etc. There is currently a need to develop a strain of Bacillus that closely represents Bacillus anthracis to be used as a BI for field applications. Bacillus thuringiensis Al Hakam is a lead candidate for this role.

Bacillus spores from various species/strains, including Bacillus anthracis, are used for laboratory bio-defense tests to develop detectors and decontaminants.

A variety of Bacillus thuringiensis strains are commercially produced as insecticides for agricultural and horticultural applications.

Spores of Bacillus anthracis vaccine strains, such as the U.S. anthrax vaccine strain Bacillus anthracis V770-NPI-R, are commercially prepared as vaccines.

There are numerous different methods that are well-known in the art utilizing both agar and broth cultures to prepare or produce spores. The limitations of agar culturing are long incubation time, high cost, and reduced safety. In addition, performance issues are associated with agar preparations because there are additional steps that may increase variability in the spore preparation, agar may be present in the final preparation, and spores grown in agar have been shown to have greater variability than broth-cultured spores.

Colony growth in layers on agar media results in uneven distribution of air and nutrients resulting in greater spore variability. Spore variability leads to subsequent variability in any application of the spores (BIs, vaccines, insecticides and laboratory testing) and lowers the confidence of any results for applications utilizing the spores.

The limitation of current broth culture protocols is the inability to achieve both high spore titer and high spore purity with few steps. That is, current broth protocols require complex, expensive purification methods.

Current methods also typically require re-inoculation or multiple inoculations, which adds manual steps and life cycles, resulting in increased mutation rates, increased time, and increased costs.

There is a need to reduce costs and time for Bacillus spore preparations. There is also a need to isolate purified, consistently sized spores in order to isolate spores as an independent variable where spores are >95% pure relative to dirt, organic debris, or other additives that are combined with spores.

### SUMMARY OF THE INVENTION

The present invention is a method of preparing different strains of Bacillus including Bacillus anthracis, Bacillus anthracis V770-NPI-R, and Bacillus thuringiensis. A protein-based sporulation broth, consisting of protein-rich or amino acid-rich nutrients (and minimized for simple sugars such as glucose), phosphate buffer and sporulation salts, is inoculated with a single inoculation of a Bacillus strain and incubated at a first temperature for a first time duration while agitated. After incubation, the protein-based sporulation broth is centrifuged to create a first spore pellet. The first spore pellet is re-suspended in a Tween 80 solution to create a wash suspension. The wash suspension is incubated for a second time duration at room temperature with agitation.

### BRIEF DESCRIPTION OF THE DRAWINGS

FIG. 1 is a table depicting titers and phase-bright percentage of Bacillus spores obtained using three different preparation protocols.

### DETAILED DESCRIPTION

For the purpose of promoting an understanding of the present invention, references are made in the text to exemplary embodiments of systems and methods for Bacillus spore preparation, only some of which are described herein. It should be understood that no limitations on the scope of the invention are intended by describing these exemplary embodiments. One of ordinary skill in the art will readily appreciate that alternate but functionally equivalent materials, components, and placement may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

Moreover, the terms “substantially” or “approximately” as used herein may be applied to modify any quantitative representation that could permissibly vary without resulting in a change in the basic function to which it is related.

By way of illustration, three exemplary embodiments of Bacillus spore preparation methods are described, each using a combination of specific, detailed steps including nutrients, temperature, time and aeration for preparing spores from different strains of Bacillus including Bacillus anthracis, Bacillus anthracis V770-NPI-R, and Bacillus thuringiensis.

Spore titers of the preparations may be evaluated as follows:
T0—Titer of culture before processing without heat shock
T1—Titer of culture before processing with a 65° C., 30 minute heat shock
T2—Titer of culture after processing, before freezing, with a 65° C., 30 minute heat shock
T3—Titer of culture after −80° C. storage and then heat shocked at 65° C., 30 minutes

Bacillus anthracis Spore Preparation

In a first exemplary embodiment, a combination of specific, detailed steps is used to prepare Bacillus anthracis spores. In further exemplary embodiments, other Bacillus spores may be prepared using the method.

Reagent Preparation

5% Nutrient Broth, consisting of 3 parts beef extract to 5 parts peptone; 30× KPO₄ Buffer; Sporulation Salts; and 0.5 M L-Glutamate are prepared as follows:

### 5% Nutrient Broth

Per Liter: 50 grams Nutrient Broth
deionized distilled water to 900 mL
adjust pH to 7.0 with NaOH
add de-ionized distilled water to 1 L volume

The broth may be aliquoted into one or more containers, e.g., glass bottles, suitable for autoclaving. The prepared broth is autoclaved for 30 minutes. If desired, the broth may be filtered through a 0.2 μm filter. Filtering can be done alone or in conjunction with autoclaving. The advantage of filtering is that it is faster, and it removes any particles that might be the same size as spores.

### 30× KPO₄ Buffer

Per Liter: 64.5 grams KH₂PO₄ (474 mM)
131.7 grams K₂HPO₄ (756 mM)
add de-ionized distilled water to 1 L volume
pH=7.0-7.2 (pH need not be adjusted if it is in this range

The buffer may be aliquoted into one or more containers, e.g., glass bottles, suitable for autoclaving. The prepared buffer is autoclaved for 30 minutes. If desired, the broth may be filtered through a 0.2 μm filter. Filtering can be done alone or in conjunction with autoclaving. The advantage of filtering is that it is faster, and it removes any particles that might be the same size as spores.

### Sporulation Salts

### Per 33.3 mL:

The following are filter sterilized (0.2 μm filters), not autoclaved:
2 mL 1M CaCl₂.2H₂O
2 mL 0.1M MnCl₂.4H₂O
2 mL 1M MgCl₂
2 mL 0.001M FeCl₃.6H₂O
2 mL 0.05M ZnCl₂

Combine all salts with 23.33 mL de-ionized distilled water. Sterile filter through a 0.2 μm filter, e.g., PES Corning filter. If desired, sporulation salts may be aliquoted, e.g., 15 mL of prepared salt solution into 15 mL Falcon tubes. Aliquots may be frozen for long-term storage, e.g., at −80° C., to prevent salt crystal formation.

### 0.5 M L-Glutamate

Per Liter: 73.6 grams glutamic acid
water to 900 mL
pH=7.0 with 10M NaOH
add de-ionized distilled water to 1 L and readjust pH if necessary
sterile filter through 0.22 μm filter
   0.5 M L-Glutamate is desirably prepared fresh at the time of use.

Media Preparation

Bacillus anthracis will sporulate well in media numbers (Nos.) 8 (0.8% Nutrient broth plus sporulation salts), 10 (2.5% Nutrient broth plus sporulation salts, 200 mM Glutamate), and 12 (2.5% Nutrient broth plus sporulation salts) with or without oil, although there is more debris if oil is added. The preferred medium for Bacillus anthracis is No. 12 without oil. Each medium is prepared as follows:

| | | | |
|---|---|---|---|
| Per Liter: | No. 12 | No. 10 | No. 8 |

| | | | | | | |
|---|---|---|---|---|---|---|
| 5% Nutrient Broth | 500 | mL | 500 | mL | 160 | mL |
| Sterile ddH₂O | 450 | mL | 50 | mL | 790 | mL |
| Sporulation Salts | 16.7 | mL | 16.7 | mL | 16.7 | mL |
| 30 × KPO₄ Buffer | 33.3 | mL | 33.3 | mL | 33.3 | mL |
| 0.5M L-Glutamate | 0 | mL | 400 | mL | 0 | mL |

Nutrient broth is combined with water, 30× KPO₄ buffer, and sporulation salts to make the protein-based sporulation broth. If using oil, 10 mL olive oil (e.g., Sigma 01514-500) per L of media (final concentration 1%) is added last and directly to the Erlenmeyer flasks. An oil amendment is one option to eliminate foaming in hydrophobic spore cultures in order to increase spore yield.

Inoculation

200 mL of medium is added per 1 L plastic baffled Erlenmeyer flask with a filtered cap (e.g., Corning Part No. 431403). Larger volumes may be used, but may not provide the same level of aeration and therefore may result in lower spore purity.

The medium is desirably pre-aerated at least 1 hour at 34° C. ±3° C., 300±20 rpm.

The medium is inoculated with approximately 1-10×10⁵ spores (heat-shocked at 65° C. for 30 minutes) per mL of sporulation media. Alternatively, Bacillus anthracis could be streaked on an agar plate such as tryptic soy agar or nutrient agar, and incubated at 34° C.±3° C. for up to 20 hours. When using a streaked plate, 1-3 colonies are sterile transferred into 10 mL of sporulation medium in a 50 mL conical tube. After vortexing for 30-60 seconds, 1 mL of inoculum is transferred into each Erlenmeyer flask with sporulation medium.

The 0.2 μm-filtered, screw-cap flask top is then installed and the flask placed in a shaker at 34° C.±3° C., 300±20 rpm for 72±2 hours. Bacillus anthracis will sporulate at other temperatures, ranging in some exemplary embodiments from 20-37° C., depending on the strain. Strains such as Pasteur require lower temperatures (25° C.) for sporulation.

Spore Preparation

Transfer one or more spore cultures to a centrifuge tube(s).

Two aliquots of 250 μL are removed from each sporulation culture dispensed into Eppendorf tubes and set aside at room temperature for subsequent titering. One aliquot will be used to titer the T0 data point and the other aliquot will be used to titer the T1 data point.

The spore cultures are centrifuged (e.g., for 10 minutes, 20° C. at 2,000×g) and the supernatant decanted.

Washes

The spore cultures are centrifuged (e.g., for 10 minutes, 20° C. at 2,000×g) and the supernatant decanted. To get additional spores out of the Erlenmeyer flasks, an equal volume of 0.1% Tween 80 may be poured into one Erlenmeyer flask, and either swirled by hand or incubated at 200-300 rpm. An equal volume means that if there was 200 mL of sporulation media in a flask, then 200 mL of 0.1% Tween 80 is added for each wash.

The Erlenmeyer flask(s) with 0.1% Tween 80 contains the first wash. This wash is transferred into the centrifuge tubes with the pelleted spores. Wash is added to just cover the spore pellet. The pellet is then resuspended by shaking in the shaker/incubator for 10 minutes and then shaking by hand, if necessary, to complete resuspension. The remainder of the 0.1% Tween 80 wash is then added to the spore sample. The centrifuge tube with spores in 0.1% Tween 80 is then placed in a shaker at room temperature, 200±20 rpm for 1 hour.

After shaking, the sample is again centrifuged and the supernatant decanted as previously described.

Another 200 mL of 0.1% Tween 80 is transferred to the Erlenmeyer flasks and a second wash is performed as described in the previous two paragraphs.

After washing, the spore pellet(s) are resuspended in 0.1% Tween 80. The volume of 0.1% Tween 80 is desirably about 10% of the sporulation volume. Thus a 200 mL spore preparation should be suspended in approximately 20 mL of 0.1% Tween 80. The pellet is dislodged again by vortexing, hand shaking, or pipetting up and down.

This should yield a final spore concentration of approximately 10⁹-10¹⁰ spores/mL.

Alternatively, the Tween 80 amendment may be varied in order to maximize spore yield and purity. In some cases, particularly for hydrophobic spores, it may be advantageous to add Tween 80 immediately after sporulation and prior to the first centrifugation (this would be the first step of spore preparation). The addition of Tween 80 prior to the first centrifugation removes spores and cells from the foam head and the Erlenmeyer flask wall. This process suspends spores and vegetative cells in solution to increase spore recovery (often by an order of magnitude) and allow vegetative cell degradation. For example, Tween 80 may be added to a sporulated culture at a final concentration of 3% and incubated at 34° C.±3° C., 300±20 rpm for 24±1 hours. Spores would be centrifuged and then washed twice with 3% Tween 80 for 1 hour each.

Two 250 μL aliquots may be transferred to Eppendorf tubes for titering the T2 data point.

The remaining spore sample may be aliquoted into Eppendorf tubes for long-term storage. Preferably, the Eppendorf tube is a pre-sterilized, 1.5 mL tube with a screw cap and an attached lid. The spores are then transferred into a freezer container such as a Styrofoam box. The tubes are desirably inverted a couple of times and then stored directly at either −70° C. or −80° C.

Titering

As previously noted, titers are obtained for the following data points:
T0—Titer of culture before processing without heat shock.
T1—Titer of culture before processing with a 65° C., 30 minute heat shock.
T2—Titer of culture after processing, before freezing, with a 65° C., 30 minute heat shock.
T3—Titer of culture after −80° C. storage and then heat shocked at 65° C., 30 minutes.

The samples are vortexed and serially diluted. The preferred diluent is either 0.1% Tween 80 or 1% 3-(N morpholino)-2-Hydroxypropanesulfonic Acid (MOPSO). The samples are then plated on tryptic soy agar (TSA) plates and incubated 16±2 hours at 34±3° C. prior to performing colony counts.

A non-diluted 100 μL spore aliquot spread on an agar plate is equivalent to a −1 (minus one) dilution or 0.1. If there are 100 CFU on this plate then there are 100/0.1=1,000 CFU per mL. A 100 μL spore aliquot combined with 900 μL of diluent is vortexed. If 100 μL of this dilution is spread on to a plate then this is a −2 (minus two) dilution or 0.01. If there are 100 CFU on this plate, then there are 100/0.01=10,000 CFU per mL.

Microscopy

The spore suspension is examined with a phase-contrast microscope or by staining to assess the quality of the spores and to determine the ratio of spores to vegetative cells (or sporangia). Desirably, phase-bright spores versus vegetative cells should be at least 95%. Spores (>10,000) would also preferably be quantified with a Beckman Coulter multisizer to demonstrate a uniformly sized spore population that is free of debris.

Bacillus anthracis V770-NPI-R Spore Preparation

In a second exemplary embodiment, a combination of specific, detailed steps is used to prepare Bacillus anthracis V770-NPI-R spores.

While this second exemplary embodiment describes a protocol useful for the preparation of U.S. anthrax vaccine strain V770-NPI-R, in further exemplary embodiment, it may also be applicable to other Bacillus strains.

Reagent Preparation

5% Nutrient Broth, 30× KPO₄ Buffer, and Sporulation Salts are prepared as previously described for Example 1.

Media Preparation

Medium No. 12 is prepared as previously described for Example 1. If desired, oil may be added directly to the flask as also previously described.

Inoculation

200 mL of media is added per 1 L plastic baffled Erlenmeyer flask with a filtered cap and pre-aerated at least 1 hour at 34° C.±3° C., 300±20 rpm as previously described for Example 1.

The medium is inoculated with approximately 1-10×10⁵ spores (heat-shocked at 65° C. for 30 minutes) per mL of sporulation media. Alternatively, Bacillus anthracis could be streaked on an agar plate such as tryptic soy agar or nutrient agar, and incubated at 34° C.±3° C. for up to 20 hours. When using a streaked plate, 1-3 colonies are sterile transferred into 10 mL of sporulation medium in a 50 mL conical tube. After vortexing for 30-60 seconds, 1 mL of inoculum is transferred into each Erlenmeyer flask with sporulation medium.

The 0.2 μm-filtered, screw-cap flask top is then installed and the flask placed in a shaker at 37° C.±3° C., 300±20 rpm for 5 hours±15 minutes.

This strain does not sporulate well at 34° C. Lower temperatures are preferable. To optimize yield, after 5 hours±15 minutes, reduce the temperature to 28° C.±3° C. Incubate at 28° C.±3° C. for an additional 67 ±2 hours so the final incubation time is 72±2 hours.

Spore Preparation

35.3 mL of 20% Tween 80 is then added to each Erlenmeyer flask to give 235.3 mL final volume and a final concentration of 3% Tween 80. Shake for 24 hours ±1 hour at 28° C.±3° C., 300±20 rpm. The addition of Tween 80 at this step does not serve as a wash. Rather, it serves to get all spores in suspension and suspend vegetative cells to allow vegetative cell degradation.

Two aliquots of 250 μL are removed from each spore culture, dispensed into Eppendorf tubes and set aside at room temperature for subsequent titering. One aliquot from each culture serves as the T0 data point. One aliquot from each culture serves as the T1 data point.

Transfer one or more flask cultures to a centrifuge tube(s).

The spore cultures are then centrifuged (e.g., for 10 minutes, 20° C. at 2,000×g) and the supernatant decanted.

Washes

To get additional spores out of the Erlenmeyer flasks, an equal volume of 3% Tween 80 may be poured into one Erlenmeyer flask, and either swirled by hand or incubated at 200-300 rpm. An equal volume means that if there was 200 mL of sporulation media in a flask, then 200 mL of 3% Tween 80 is added for each wash.

The Erlenmeyer flask(s) with 3% Tween 80 contains the first wash. This wash is transferred into the centrifuge tubes with the pelleted spores. Wash is added to just cover the spore pellet. The pellet is then resuspended by shaking in the shaker/incubator for 10 minutes and then shaking by hand, if necessary, to complete resuspension. The remainder of the 3% Tween 80 wash is then added to the spore sample. The centrifuge tube with spores in 3% Tween 80 is then placed in a shaker at room temperature, 200±20 rpm for 1 hour.

After shaking, the sample is again centrifuged and the supernatant decanted.

Another 200 mL of 3% Tween 80 is transferred to the Erlenmeyer flasks and a second wash is performed as described in the previous two paragraphs.

After the two 3% Tween 80 washes, the spore pellet(s) are resuspended in 0.1% Tween 80. The volume of 0.1% Tween 80 is desirably about 10% of the sporulation volume. Thus a 200 mL spore preparation should be suspended in 20 mL of 0.01% Tween 80. The pellet is dislodged again by vortexing, hand shaking or pipetting up and down.

The final spore concentration should be approximately 10⁹-10¹⁰ spores/mL.

Two 250 μL aliquots may be transferred to Eppendorf tubes for titering the T2 data point.

The remaining spore sample may be aliquoted into Eppendorf tubes for long-term storage. Preferably, the Eppendorf tube is a pre-sterilized, 1.5 mL tube with a screw cap and an attached lid. The spores are then transferred into a freezer container such as a Styrofoam box. The tubes are desirably inverted a couple of times and then stored directly at either −70° C. or −80° C.

Titering

Titers are obtained for T0, T1, T2, and T3 as previously described for Example 1. Serial dilutions and colony counts are performed as also previously described for the first exemplary embodiment.

Microscopy

The spore suspension is examined as previously described for Example 1. Desirably, phase-bright spores versus vegetative cells should be at least 95%.

Bacillus thuringiensis Spore Preparation

In a third exemplary embodiment, a combination of specific, detailed steps is used to prepare Bacillus thuringiensis spores.

While this third exemplary embodiment is described for Bacillus thuringiensis spore preparation, the exemplary protocol may also be used for Bacillus anthracis and other Bacillus spores; however, the yield may be lower than the protocol described in the first exemplary embodiment.

Reagent Preparation

5% Nutrient Broth, 30× KPO₄ Buffer, and Sporulation Salts are prepared as previously described for in the first exemplary embodiment.

Media Preparation

Medium No. 8 is prepared as previously described for the first exemplary embodiment. If desired, oil may be added directly to the flask as also previously described for the first exemplary embodiment.

Inoculation

200 mL of media is added per 1° L plastic baffled Erlenmeyer flask with a filtered cap and pre-aerated at least 1 hour at 34° C.±3° C., 300±20 rpm as previously described for the first exemplary embodiment.

The medium is inoculated with approximately 1-10×10⁵ spores (heat-shocked at 65° C. for 30 minutes) per mL of sporulation media. Alternatively, Bacillus anthracis could be streaked on an agar plate such as tryptic soy agar or nutrient agar, and incubated at 34° C.±3° C. for up to 20 hours. When using a streaked plate, 1-3 colonies are sterile transferred into 10 mL of sporulation medium in a 50 mL conical tube. After vortexing for 30-60 seconds, 1° mL of inoculum is transferred into each Erlenmeyer flask with sporulation medium.

The 0.2 μm filtered, screw-cap flask top is then installed and the flask placed in a shaker at 34° C.±3° C., 300±20 rpm for 72±2 hours. Many Bacillus thuringiensis strains sporulate well between 28-37° C.

Spore Preparation

35.3 mL of 20% Tween 80 is then added to each Erlenmeyer flask to give 235.3 mL final volume and a final concentration of 3% Tween 80. Shake for 24 hours ±1 hour at 28° C.±3° C., 300±20 rpm. The addition of Tween 80 at this step does not serve as a wash. Rather, it serves to get all spores in suspension and suspend vegetative cells to allow vegetative cell degradation.

Two aliquots of 250 μL are removed from each spore culture, dispensed into Eppendorf tubes and set aside at room temperature for subsequent titering. One aliquot from each culture serves as the T0 data point. One aliquot from each culture serves as the T1 data point.

Transfer one or more flask cultures to a centrifuge tube(s).

Centrifuge (e.g., for 10 minutes, 20° C. at 2,000×g) and then decant the supernatant.

Washes

To get additional spores out of the Erlenmeyer flasks, an equal volume of 3% Tween 80 may be poured into one Erlenmeyer flask, and either swirled by hand or incubated at 200-300 rpm. An equal volume means that if there was 200 mL of sporulation media in a flask, then 200 mL of 3% Tween 80 is added for each wash.

The Erlenmeyer flask(s) with 3% Tween 80 contains the first wash. This wash is transferred into the centrifuge tubes with the pelleted spores. Wash is added to just cover the spore pellet. The pellet is then resuspended by shaking in the shaker/incubator for 10 minutes and then shaking by hand, if necessary, to complete resuspension. The remainder of the 3% Tween 80 wash is then added to the spore sample. The centrifuge tube with spores in 3% Tween 80 is then placed in a shaker at room temperature, 200±20 rpm for 1 hour.

After shaking, the sample is again centrifuged and the supernatant decanted.

Another 200 mL of 3% Tween 80 is transferred to the Erlenmeyer flasks and a second wash is performed as described in the previous two paragraphs.

After the two 3% Tween 80 washes, the spore pellet(s) are resuspended in 0.1% Tween 80. The volume of 0.1% Tween 80 is desirably about 10% of the sporulation volume. Thus a 200 mL spore preparation should be suspended in 20 mL of 0.1% Tween 80. The pellet is dislodged again by vortexing, hand shaking or pipetting up and down.

The final spore concentration should be approximately 10⁹-10¹⁰ spores/mL.

Two 250 μL aliquots may be transferred to Eppendorf tubes for titering the T2 data point.

The remaining spore sample may be aliquoted into Eppendorf tubes for long-term storage. Preferably, the Eppendorf tube is a pre-sterilized, 1.5 mL tube with a screw cap and an attached lid. The spores are then transferred into a freezer container such as a Styrofoam box. The tubes are desirably inverted a couple of times and then stored directly at either −70° C. or −80° C.

Titering

Titers are obtained for T0, T1, T2, and T3 as previously described for Example 1. Serial dilutions and colony counts are performed as also previously described for the first exemplary embodiment.

Microscopy

The spore suspension is examined as previously described for Example 1. Desirably, phase-bright spores versus vegetative cells should be at least 95%.

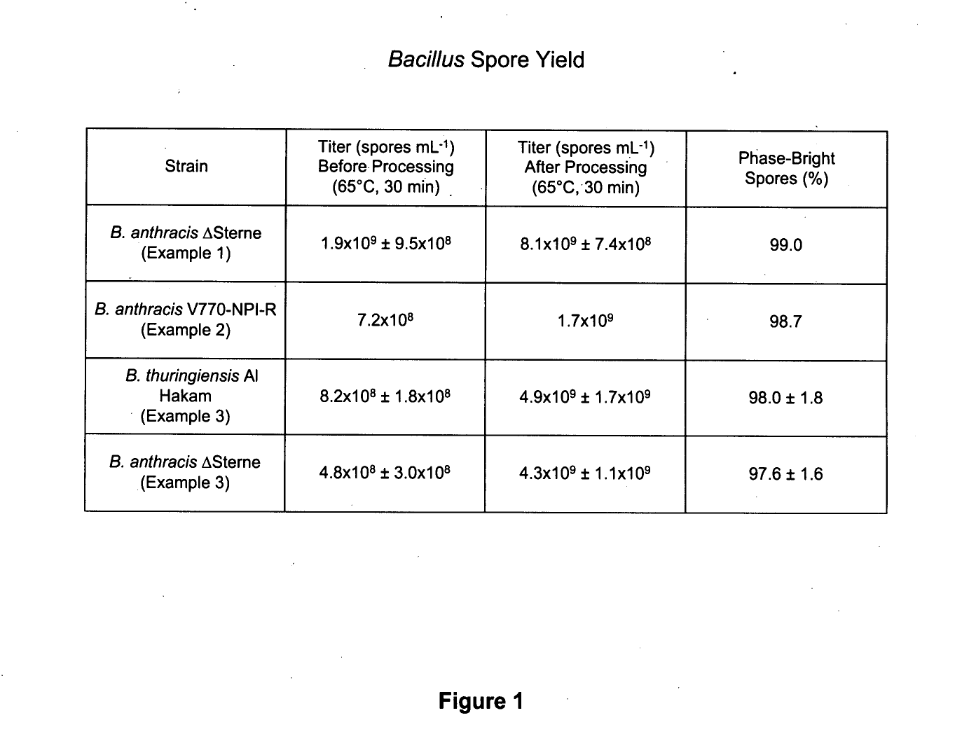

FIG. 1 shows the titers and phase-bright percentage of Bacillus spores according to the three different examples. As FIG. 1 illustrates, all three examples meet or exceed desirable criteria of:
1. Threshold yield of 10⁸ spores-mL⁻¹ of sporulation media (sporulation yield prior to processing)
2. Heat resistance at 65° C. for 30 minutes (demonstrates that spores are viable)
3. >95% phase-bright spores

The three exemplary embodiments detail procedures that consistently provide high titers of consistently sized spores meeting the above criteria. The advantages of these procedures were reduced cost, reduced time, improved performance (less variability in spore size), and improved safety (fewer hands-on steps). Spores were isolated with fewer steps, in less time, and in higher purity than conventional methods.

In the exemplary embodiments described, spore suspensions are prepared using flasks. For example, up to 300 mL may be prepared using a 1 L flask, and up to 1 L may be prepared in a 3 L flask. In still further exemplary embodiments, larger containers other than flasks may be used to create larger volumes of spore suspensions. In some exemplary embodiments, bioreactors/fermentors known in the art may be used, and can be used to create up to 10 L of spore suspension.

In still further exemplary embodiments, containers such as pressure cookers and kettles may be used to prepare spore suspensions. For example, up to 10 L of spore suspension may be prepared in a 16 L pressure cooker and up to 300 L in a 400 L kettle. In some exemplary embodiments, pressure cookers, kettles and other containers may need to be physically modified in order to be used to create spore suspensions. For example, containers may be modified with aeration apertures, lids able to withstand pressurization, latches, air compressor connections, manual agitation devices, motorized agitation devices, and combinations of these and other modifications necessary to adapt the container for creating spore suspensions.

When using different sizes of containers to prepare varying volumes of spore suspensions, different methods for agitating and aerating the solution may be used. For example, when creating small volumes of spore suspension, such as when using a 1 L flask, the solution make be shaken, and air passively introduced to the solution through a permeable membrane at the top of the flask. In such embodiments, agitation and aeration are simultaneous.

In further exemplary embodiments, when using a larger container, such as a 3 L flask, a stir bar or stir rod may be used and air or oxygen may be introduced by forcing the air or oxygen into the solution under pressure. Larger spore suspension volumes may require stir paddles or other mechanisms or devices to properly agitate the suspension. When aerating larger containers, oxygen and air may be introduced to the container using a hose to pressurize the container. In some exemplary embodiments, a HEPA (High-Efficiency Particulate Air) filter may be used to limit or eliminate the amount of contaminants introduced to the spore suspension.

1. A method of preparing Bacillus spores with a consistent protein-based sporulation broth comprising the steps of:
preparing a medium comprised of protein-rich nutrients, a PO₄ buffer, and sporulation salts;
performing a single inoculation of said medium with a live Bacillus strain;
incubating said inoculated medium at a first temperature for a first time duration while agitated at 280 to 320 revolutions-per-minute (rpm);
centrifuging said inoculated medium for 10 minutes at 20° C. and 2000×g to create a first spore pellet;
re-suspending said first spore pellet in a first Tween 80 solution to create a wash suspension; and
incubating said wash suspension for a second time duration at room temperature while agitated at 280 to 320 rpm.

2. The method of claim 1 wherein said first temperature is 25° C. to 38° C., and said first time duration is 70 hours to 74 hours.

3. The method of claim 1 wherein said first temperature is 34° C. to 40° C. and said first time duration is 4 hours and 45 minutes to 5 hours and 15 minutes.

4. The method of claim 3 which further includes the steps of:
incubating said inoculated medium at a second temperature of 25° C. to 31° C. for a third time duration of 65 hours to 69 hours with agitation at 280 to 320 rpm to produce a media suspension;
adding Tween 80 to said media suspension to create a suspension solution; and
incubating said suspension solution at a third temperature of 31° C. to 37° C. at a rotational speed of 280 to 320 rpm for a fourth time duration of 23 hours to 25 hours to reconstitute said inoculated medium.

5. The method of claim 1 wherein said second time duration is 55 minutes to 65 minutes.

6. The method of claim 1 which further includes the steps of:
adding Tween 80 to said inoculated medium; and
incubating said inoculated medium at 31° C. to 37° C. for 23 to 25 hours with agitation.

7. The method of claim 1 which further includes the step of centrifuging said wash suspension to create a second spore pellet.

8. The method of claim 7 which further includes the step of washing said second spore pellet with a volume of a second Tween 80 solution.

9. The method of claim 8 wherein said second Tween 80 solution is selected from the group consisting of a 3% Tween 80 solution and a 0.1% Tween 80 solution.

10. The method of claim 9 which further includes the step of re-suspending said second spore pellet in a volume of 0.1% Tween 80 solution.

11. The method of claim 1 which further includes the step of evaluating spore preparation by titering.

12. The method of claim 1 which further includes the step of evaluating spore preparation by microscopy and Coulter analysis.

13. The method of claim 1 wherein said buffer is a phosphate buffer.

14. The method of claim 1 wherein said buffer is a KPO₄ buffer.

15. The method of claim 1 wherein said first Tween 80 solution is selected from the group consisting of a 3% Tween 80 solution and a 0.1% Tween 80 solution.

16. The method of claim 1 wherein the concentration of protein-based broth in said medium is 2.5% Nutrient Broth.

17. The method of claim 1 wherein the concentration of protein-based broth in said media is 0.8% Nutrient Broth.

18. The method of claim 1 wherein said protein-based broth includes a manganese supplement.

19. The method of claim 1 wherein said media further includes glutamate.

20. The method of claim 19 wherein the concentration of glutamate in said media is 200 mM.

21. The method of claim 1 wherein said sporulation broth is selected from the group consisting of CaCl₂.2H₂O, MnCl₂.4H₂O, MgCl₂, FeCl₃.6H₂O, ZnCl₂, and combinations thereof.

22. The method of claim 1 wherein said sporulation broth comprises 1 mM CaCl₂.2H₂O, 0.1 mM MnCl₂.4H₂O, 1 mM MgCl₂.0.001 mM FeCl₃.6H₂O, and 0.05 mM ZnCl₂.

23. The method of claim 1 wherein the Bacillus inoculated is Bacillus spores.

24. The method of claim 23 which further includes the step of incubating said spores at 65° C. for 30 minutes to heat shock the spores prior to inoculating the media.

25. The method of claim 1 wherein said media is inoculated with 1-10×10⁵ Bacillus spores per mL of media.

26. The method of claim 1 wherein said Bacillus is selected from the group consisting of Bacillus anthracis, Bacillus anthracis V770-NPI-R, and Bacillus thuringiensis.

27. The method of claim 1 wherein the Bacillus inoculated is vegetative Bacillus cells less than 20 hours old.
